(19) United States
(12) Patent Application Publication
(10) Pub. No.: US 2004/0192888 A1
(43) Pub. Date: Sep. 30, 2004

(54) Method of preparing peptide with high yield and high purity using2-(4-nitrophenylsulfonyl) ethoxycarbonyl-amino acids

(76) Inventors: Hack-Joo Kim, Seoul (KR); YoungDeug Kim, Kyonggi-do (KR); Chanyoung Ko, Kyonggi-do (KR)

Correspondence Address: BOZICEVIC, FIELD & FRANCIS LLP, 200 MIDDLEFIELD RD, SUITE 200, MENLO PARK, CA, 94025, US

(21) Appl. No.: 10/479,907

(22) PCT Filed: Jun. 5, 2002
(86) PCT No.: PCT/KR02/01070

(30) Foreign Application Priority Data
Jun. 7, 2001 (KR) 2001-0031768

(51) Int. Cl. C07K 1/02

(52) U.S. Cl. 530/333

(57) ABSTRACT

Disclosed is a method of synthesizing high yield and high purity peptides with desired target amino acid sequences from solid phase within a short time period, using 2-(4-nitrophenylsulfonyl)ethoxycarbonyl-amino acids (Nsc-amino acids).

### TECHNICAL FIELD

The present invention pertains to a method of preparing a target peptide having high yield and high purity within a short time period, by solid phase peptide synthesis using 2-(4-nitrophenylsulfonyl)ethoxycarbonyl-amino acids (Nsc-amino acids).

### BACKGROUND ART

Typically, solid phase peptide synthesis is widely employed for the preparation of biologically active peptides which are used in medical and biological research and also as active substances in pharmaceutical, veterinary, agriculture, fishery, environmental and diagnostic fields.

The fundamental principle of solid phase peptide synthesis can be outlined as a stepwise elongation of a peptide chain by means of repeated cycles of chemical reactions, starting from the first C-terminal amino acid attached to an insoluble carrier. During the course of the synthesis, target products of all reactions remain bound to the carrier, whereas excess reactants and by-products are removed by filtration and washing of the carrier.

In order to perform the solid phase synthesis of a peptide, the first amino acid (C-terminal of the target amino acid sequence) with a protected amino group, through a free carboxyl group thereof, forms an amide or ester bond with an amino or hydroxyl group of an anchor group linked to a polymeric carrier, to give a protected aminoacyl-polymer. Then such protected aminoacyl-polymer is deprotected by treatment of a basic reagent, and the aminoacyl-polymer with a free amino group is formed. To the free amino group of this polymer, a protected amino acid monomer is acylated, thereby giving the protected dipeptidyl-polymer. Thereafter, the synthetic cycles, which consist of the steps of deprotection and acylation, are repeated until the peptide having the target amino acid sequence is obtained. Finally, the terminal protective group of the synthesized peptide is deprotected, and the peptide is detached from the insoluble carrier.

In practical solid phase synthesis, excessive molar amounts (2 to 10-fold) of acylating reagents are usually employed to assure complete conversion, therefore, all functional groups in side chains of the amino acids, such as amino, carboxyl, hydroxyl, thiol and guanidino groups, should be blocked with appropriate protective groups. Such protective groups for this purpose must be selected carefully to provide reliable and permanent protection of the side chains under conditions of peptide-polymer acylations and during the cleavage of temporary protective group.

As such general protective groups, use has been made of N_{α}-tert-butoxycarbonyl (Boc) group deprotected by the action of acidic reagents, 1-(3,5-tert-butylphenyl)-1-methyl-ethoxycarbonyl (t-Bumeoc) group deprotected by the action of acidic reagents of medium strength, N_{α}-9-fluorenylmethoxycarbonyl (Fmoc) group, which is resistant to acidic reagents and deprotected by organic bases in aprotic solvents, Fmoc/tBu using tert-butoxycarbonyl (tBU) deprotected by the action of acids as a side chain, etc. The solid phase peptide synthesis using amino acids protected by the Fmoc group has been widely used.

However, Fmoc-amino acids suffer from the disadvantages of instability to weak bases or neutral solvents, incomplete deprotection even in the presence of high concentrations of piperidine with respect to the peptide having long amino acid sequence, insufficient trapping of dibenzofulvene produced upon cleavage of Fmoc, low solubility in most solvents used in the solid phase synthesis due to its hydrophobic portions, and low acylation yield.

Thus, there is required need for development of protective groups which can solve such problems. The present inventors have developed, as a protective group of amino acids, 2-(4-nitrophenylsulfonyl)ethoxy carbonyl (Nsc) group represented by the following formula I, and applied for patent of such group (Korean Patent No. 241948).

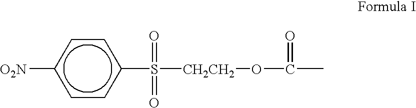

The Nsc that has a nitro group and a sulfonyl group therein is responsible for increasing solubility of amino acids, and preventing interaction of hydrophobic portions between peptide chains. In particular, Nsc group is usefully employed for peptide synthesis, since a mechanism for removing such protective group is similar to a deprotection mechanism of Fmoc group.

### DISCLOSURE OF INVENTION

Accordingly, the intensive and thorough research on protective groups suitable for solid phase peptide synthesis as well as a synthesis method to obtain high yield and purity of peptide within a short time period, carried out by the present inventors aiming to avoid the problems encountered in the prior arts, resulted in the finding that, when solid phase synthesis is performed using Nsc-amino acids as protected amino acids and the acylating step is carried out using dichloromethane as an acylation solvent in the temperature range of from 20 to 50° C., the above purpose can be achieved, and also Nsc-amino acids, stable in dichloromethane used as the acylation solvent, can be stored in a solution state dissolved in dichloromethane, with no need for preparation of Nsc-amino acids for each synthesis.

Thus, it is an object of the present invention to provide a method of preparing a peptide having a desired amino acid sequence with high yield and high purity from a solid phase, during a shorter time period.

It is another object of the present invention to provide a method of stably storing Nsc-amino acids used for solid phase peptide synthesis.

In accordance with an embodiment of the present invention, there is provided a method of preparing a peptide comprising the steps of: forming a protected aminoacyl-polymer onto an insoluble carrier through a free carboxyl group of a protected amino acid; deprotecting the protected aminoacyl-polymer; acylating a protected amino acid monomer to a free amino group of the deprotected polymer; repeating the deprotection and the acylation steps until a target amino acid sequence-containing peptide is synthesized; and detaching the synthesized peptide from the insoluble carrier after deprotection of the terminal protective group in the synthesized peptide, wherein 2-(4-nitrophenylsulfonyl) ethoxycarbonyl-amino acids (Nsc-amino acids) are used as the protected amino acid, and the acylating step is performed using a dichloromethane solvent at 20-50° C.

In accordance with another embodiment of the present invention, there is provided a method of storing Nsc-amino acids comprising storing 2-(4-nitrophenylsulfonyl) ethoxycarbonyl-amino acids (Nsc-amino acids) in a solution state dissolved in dichloromethane.

The Nsc-amino acids used in the present invention, which are represented by the following formula I, are produced by treating the amino acid represented by the following formula II in a mixed solvent of water/organic solvent with 2-(nitrophenylsulfonyl)chloroformate represented by the following formula III at 0-40° C., preferably at 0-20° C., in the presence of a base:

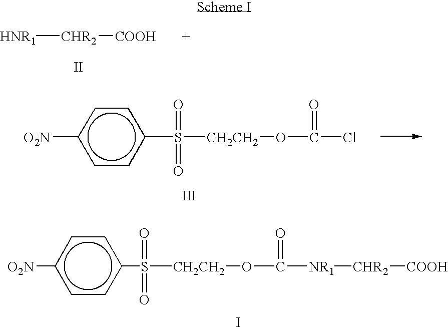

Wherein,

R₁ represents a hydrogen atom; and

R₂ represents hydrogen, methyl, isopropyl, 1-methylpropyl, 2-methylpropyl, t-butoxymethyl, 1-t-butoxyethyl, 2-methylthioethyl, benzyl, carboxamidomethyl, 2-carboxamidomethyl, t-butoxycarbonylmethyl, 2-(t-butoxycarbonyl)ethyl, 4-(t-butoxycarbonyl)ethyl, 4-(4-butoxycarbamido)butyl, 4-t-butoxybenzyl, indolyl-3-methyl, S-(triphenylmethyl)thiomethyl, 1-(triphenylmethyl) imidazolyl-4-methyl, 3-(N^{G}-mesitylenesulfonyl) guanidinopropyl, N-xanthylcarboxamidomethyl, 2-(N-xanthylcarboxamido)ethyl or S-(acetamidomethyl)thiomethyl; or

R₁ and R₂ together represent a propylene radical.

Thusly prepared Nsc-amino acids of the formula I are crystalline compounds, which are insoluble or slightly soluble in water and soluble in polar organic solvents, and are stable for long-term storage in the temperature range of −10 to 25 ° C.

According to the present invention, the solid phase peptide synthesis by use of the Nsc-amino acids of the formula I is as follows:

(1) Formation Step of Protected Aminoacyl-polymer to Insoluble Carrier

The first Nsc-amino acid (C-terminal of the target amino acid sequence) is covalently attached to an insoluble polymeric carrier by an ester or amide bond formed through the free carboxyl group, to give Nsc-aminoacyl-polymer.

A variety of polymers may be used as a polymeric carrier, such as cross-linked or porous polystyrene, cross-linked poly-N,N-dimethylacrylamide in granular form or as a composite with kieselgel, cross-linked dextrans, celluloses, papers and other polymers known in the art.

For the attachment of the first Nsc-amino acid, the polymeric carrier should contain appropriate anchor groups. In most instances, preferable anchor groups are those which provide the cleavage of synthesized peptide from the insoluble carrier with the liberation of C-terminal carboxyl or carboxamide group during treatment of peptidyl-polymer with acidic reagents, such as trifnoroacetic acid and its solutions or hydrogen chloride solutions in an organic solvent. Such anchor groups for the ester type attachment are exemplified by 4-hydroxymethylphenoxyalkyl, 4-chloro- or 4-bromo-methylphenoxyalkyl, (α-hydroxydiphenylmethyl and other groups known in the art; for the carboxamido type attachment there may be known di- and tri-alkoxybenzhydrylamine groups, 4-aminomethyl-3,5-dimethoxyphenoxyalkyl group and other known groups employed for this purpose.

Attachment of C-terminal Nsc-amino acid on an anchor group to the insoluble carrier can be carried out through a known method in the art.

(2) Deprotection Step

With a view to cleaving the protective group from the prepared Nsc-aminoacyl polymer, the protected aminoacyl polymer is treated with a basic reagent. Examples of the preferred basic reagent include nitrogen type bases, for instance, ammonia, morpholine, piperidine, piperazine, diethylamine, 1,8-diazabicyclo[5,4,0]undec-7-ene, 1,1,3,3-tetramethylguanidine and solutions thereof in aprotic organic solvents. More preferable basic reagent is 20-50% solution of piperidine in DMF.

As such, the Nsc-group is cleaved with the formation of N-[2-(nitrophenylsulfonyl)ethyl]piperidine and carbon dioxide, and an amino group is liberated.

(3) Acylation Step

The aminoacyl-polymer having the free amino group is acylated with the Nsc-amino acid, thus giving Nsc-dipeptidyl-polymer.

An acylating reagent includes, but is not limited to, 4-nitrophenyl, pentachlorophenyl, pentafluorophenyl, 1-hydroxybenzotriazolyl esters of Nsc-amino acids, and other known types of active esters used in solid phase peptide synthesis; or symmetric anhydrides of Nsc-amino acids.

Acylation can also be performed by use of Nsc-amino acids in the presence of known coupling reagents, e.g. dicyclohexylcarbodiimide, diisopropylcarbodiimide, and benzotriazolyl-1-oxy-(tris-dimethylamino)phosphonium hexafluorophosphate.

In the inventive method, dichloromethane is used as the acylating solvent. The desired peptide can be obtained in higher yield and purity when Nsc-amino acid and dichloromethane are used, compared with when Fmoc-amino acids and dimethylformamide, as the protected amino acid and as the acylating solvent, respectively.

As for reaction time, since a synthetic method using conventional Fmoc-amino acid/DMF is performed at room temperature, the reaction time takes 1 hour or longer. But when dichloromethane is used as the acylating solvent and the reaction is conducted at high temperatures of 20-50° C., the reaction is completed within from 5 minutes to 1 hour, depending on a kind of amino acid to be added to the peptide synthesis. So, the desired peptide can be produced within shorter time period.

(4) Peptide Synthesis Step

A synthesis cycle consisting of the steps of deprotection and acylation is repeated until the assembly of the peptide having the desired amino acid sequence is completed.

(5) Deprotection and Detachment Step of Target Peptide from Insoluble Carrier

The desired Nsc-peptidyl-polymer is condensed, after which the terminal protective group is cleaved in the manner as described above, and the peptide is detached from the anchor group of the insoluble carrier.

For this purpose, the acidic reagents, which are known in the art to cleave tert-butyl type protective groups, are exemplified by trifluoroacetic acid, solutions of methanesulfonic or para-toluenesulfonic acid, containing or not containing known additives for trapping evolved carbonium ions, such as water, anisole, thioanisole, dimethylsulfide, and ethanedithiol-1,2,-triisopropylsilane.

As for peptide synthesis, in comparison to Fmoc-amino acids prepared for each synthetic reaction, Nsc-amino acids need not be prepared upon every synthesis. Since Nsc-amino acids are stable in a solution state dissolved in dichloromethane, such solutions can be stored at room temperature or refrigeration temperature.

### BRIEF DESCRIPTION OF DRAWINGS

The above and other objects, features and other advantages of the present invention will be more clearly understood from the following detailed description taken in conjunction with the accompanying drawings, in which:

FIG. 1 is a profile showing the result of reverse phase high pressure liquid chromatography (HPLC) of a peptide (Tyr12), which is a tyrosine polymer prepared by acylating Nsc-Tyr(tBu)-OH as a protected amino acid using dichloromethane as an acylation solvent at 40° C.

FIG. 2 is a profile showing the result of reverse phase HPLC of a peptide (Tyr12), which is a tyrosine polymer prepared by acylating Fmoc-Tyr(tBu)-OH as a protected amino acid using dimethylformamide as an acylation solvent at room temperature.

FIG. 3 is a profile showing the result of reverse phase HPLC of a peptide (salmon calcitonin), prepared by acylating an Nsc-amino acid as a protected amino acid using dichloromethane as an acylation solvent at 40° C.

FIG. 4 shows a profile showing the result of reverse phase HPLC of a peptide (salmon calcitonin), prepared by acylating a Fmoc-amino acid as a protected amino acid using dimethylformamide as an acylation solvent at room temperature.

### BEST MODES FOR CARRYING OUT THE INVENTION

A better understanding of the present invention may be obtained in light of the following examples which are set forth to illustrate, but are not to be construed to limit the present invention.

### TEST EXAMPLE 1

### Dissolution Comparison of N_{α}-Nsc-Amino Acids and N_{α}-Fmoc-amino acids

Since only amino acids fully dissolved in a solvent can be used in acylation reactions, the following tests were performed to find solvent conditions in which amino acids can be completely dissolved.

100 mg of each of N_{α}-Nsc-amino acids and N_{α}-Fmoc-amino acids shown in Table 1, below, were precisely weighed and added to two containers added with 1 ml of dichloromethane (hereinafter, referred to as “DCM”), followed by stirring for 5 minutes. The dissolved states of amino acids were observed and are expressed in column A of the following Table 1.

Other amino acids, exclusive of totally dissolved amino acids, were heated in an oven at 40° C. for 5 minutes, and allowed to stand at room temperature for 5 minutes. Their dissolved states are shown in column B.

As such, the undissolved amino acids were dissolved in 15% dimethylformamide (hereinafter, referred to as “DMF”)/DCM in the same manner as in the case of the above column A, and their dissolved states are presented in column C.

Still undissolved amino acids were heated in an oven at 40° C. for 5 minutes, and allowed to stand at room temperature for 5 minutes. Their dissolved states were observed and are shown in column D.

TABLE 1

| | Amino | Nsc | | | | Fmoc | | | |
|---|---|---|---|---|---|---|---|---|---|
| | Acid | A | B | C | D | A | B | C | D |
| 1 | Ala | X | X | ◯ | ◯ | X | X | Δ | ◯ |
| 2 | Gly | X | X | Δ | ◯ | X | X | Δ | ◯ |
| 3 | Iie | Δ | ◯ | ◯ | ◯ | Δ | Δ | ◯ | ◯ |
| 4 | Leu | X | X | Δ | ◯ | Δ | ◯ | ◯ | ◯ |
| 5 | Met | Δ | ◯ | ◯ | ◯ | X | ◯ | ◯ | ◯ |
| 6 | Phe | X | X | ◯ | ◯ | X | X | Δ | ◯ |
| 7 | Pro | ◯ | ◯ | ◯ | ◯ | ◯ | ◯ | ◯ | ◯ |
| 8 | Trp | X | X | Δ | ◯ | X | X | ◯ | ◯ |
| 9 | Val | ◯ | ◯ | ◯ | ◯ | X | X | ◯ | ◯ |
| 10 | Cys | ◯ | ◯ | ◯ | ◯ | X | X | Δ | ◯ |
| 11 | His | Δ | ◯ | ◯ | ◯ | ◯ | ◯ | ◯ | ◯ |
| 12 | Asn | X | X | ◯ | ◯ | X | X | ◯ | ◯ |
| 13 | Gln | X | X | ◯ | ◯ | ◯ | ◯ | ◯ | ◯ |
| 14 | Asp | ◯ | ◯ | ◯ | ◯ | X | X | ◯ | ◯ |
| 15 | Glu | ◯ | ◯ | ◯ | ◯ | ◯ | ◯ | ◯ | ◯ |
| 16 | Ser | ◯ | ◯ | ◯ | ◯ | ◯ | ◯ | ◯ | ◯ |
| 17 | Thr | ◯ | ◯ | ◯ | ◯ | ◯ | ◯ | ◯ | ◯ |
| 18 | Tyr | ◯ | ◯ | ◯ | ◯ | Δ | ◯ | ◯ | ◯ |
| 19 | Lys | ◯ | ◯ | ◯ | ◯ | ◯ | ◯ | ◯ | ◯ |
| 20 | Arg | ◯ | ◯ | ◯ | ◯ | ◯ | ◯ | ◯ | ◯ |

Note:
◯: dissolution,
Δ: partial dissolution,
X: no dissolution

From the above table, it can be seen that Nsc-amino acids are better dissolved in dichloromethane than Fmoc-amino acids, and both of Nsc- and Fmoc-amino acids have excellent dissolution in a mixed solvent of 85/15 dichloromethane/dimethylformamide, and dissolution is improved upon heating to 40° C.

### TEST EXAMPLE 2

### Volume Change of Resin in Solvent

As an insoluble carrier, two 1 g fractions of each of polystyrene resin and polyethyleneglycol (PEG) resin were prepared, and contained in four marked 25 ml graduated cylinders. 25 ml of DCM and 25 ml of DMF were added to each of two polystyrene-filled cylinders and each of two PEG-filled cylinders, and allowed to stand for 10 minutes. Thereafter, the volume of the swollen resin was measured. The results are shown in Table 2, below.

TABLE 2
Swelling Power of Resin

| Resin | DCM (ml) | DMF (ml) |
|---|---|---|
| Polystyrene resin | 7.9 | 6.5 |
| PEG resin | 6.0 | 5.8 |

From the above table 2, it can be seen that polystyrene resin mainly used as the insoluble carrier in solid phase peptide synthesis is better swelled in dichloromethane (DCM). So, in the case of using DCM as the acylating solvent, it appears that the reactions of acylation, detachment, or washing after reaction completion, can be effectively performed.

### EXAMPLE 1

### Synthesis of Dodeca Tyrosine Peptide

a) Introduction of Polymeric Terminal Group

To 3 ml of DCM was added 250 mg of benzyloxybenzyl-alcohol styrene-1% divinylbenzene copolymer (1.0 meq, OH/g), and then 0.7 mmol Nsc-tyrosine and 0.35 mmol diisopropyl carbodiimide. 0.1 mmol 4-dimethylaminopyridine was further added thereto and stirred at room temperature for 24 hours. The reaction was filtered, washed with DCM and then with ethanol, ether and hexane, and dried with a vacuum dryer using phosphorous pentoxide as an absorbent for 24 hours, to give Nsc-tyrosine-resin polymer, 400 mg.

b) Condensation of Peptide

The above Nsc-tyrosine-resin polymer (200 mg) was charged into a 10 ml polypropylene reactor, washed with DMF and subjected to the following synthesis protocol:

1. prewashing: 33% piperidine/DMF, 4 ml:0.5 minutes

2. deprotection: 33% piperidine/DMF, 4 ml:15 minutes

3. washing: DMF, 3×(4 ml: 1 minute); DCM, 3×(4 ml: 1 minute)

4. acylation: N_{α}-Nsc-tyrosine, 0.5 mmol; benzotriazolyl-1-oxo-(dimethylamino) phosphonium hexafluorophosphate, 0.5 mmol; 1-hydroxybenzotriazol, 0.5 mmol; diisopropylimide, 1 mmol; DCM, 3 mmol: 10 minutes (40° C.)

5. washing: DCM, 5×(4 ml: 1 minute).

Such reaction cycle was repeated 11 times to synthesize polytyrosine-resin polymer having 12 residues.

The polytyrosine-resin polymer was treated with 33% piperidine/DMF (4 ml) for 20 minutes and washed with DMF and DCM, and then ethanol, ether and hexane.

c) Deprotection and Detachment of Peptide

The polytyrosine-resin polymer was added to 5 ml of a mixture comprising 95% trifluoroacetic acid, 2.5% water, 2.5% triisopropylsilane, and shaken at room temperature for 60 minutes. Such reaction was filtered, washed with 2 ml trifluoroacetic acid, and the filtrate combined with washings was diluted with 100 ml of cold absolute ether, and the precipitate was filtered and washed with ether, followed by drying under vacuum, to give crude tyrosine polymer, 170 mg (yield: 85%).

d) Analysis

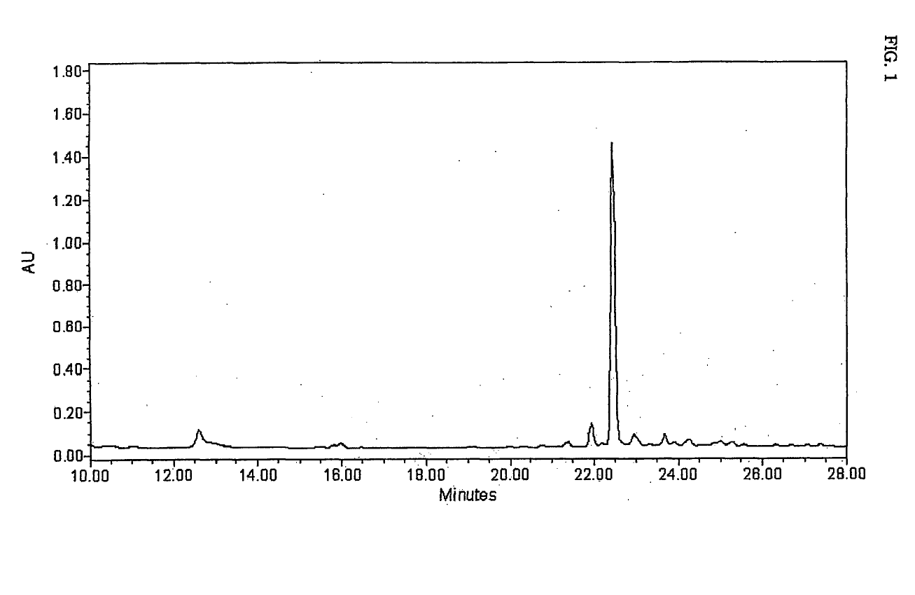

The crude tyrosine polymer was dissolved in 1 ml of DMF and analyzed by reverse phase HPLC using DISOGEL RP-C18 (Disogel, Japan 4.5×250 ml), to obtain a profile (purity 89%) as shown in FIG. 1. As for other dodeca peptides, fractions containing pure peptide were collected and analyzed using a mass analyzer, to obtain M+H 1999, the desired value.

### COMPARATIVE EXAMPLE 1

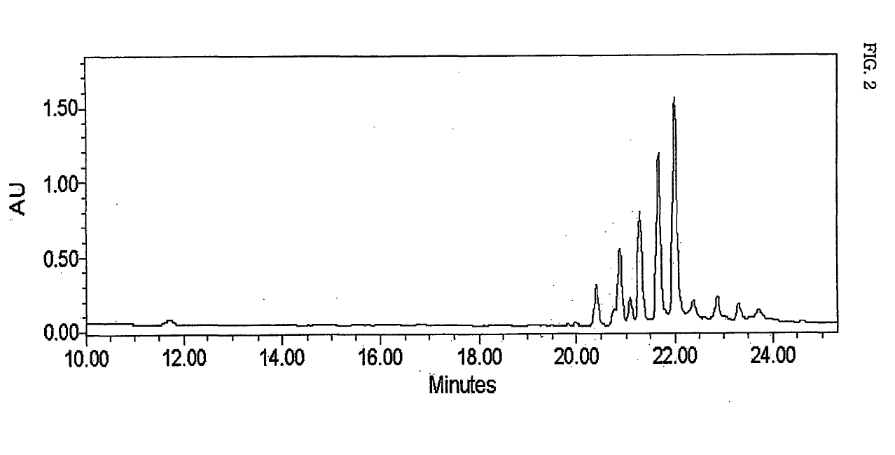

According to the solid phase peptide synthesis using a known Fmoc amino acid, except that acylation was performed at room temperature for 1 hour using Fmoc-tyrosine as an amino acid and DMF as an acylation solvent, the crude tyrosine polymer (yield: 80%), 160 mg, was produced, which was then analyzed by reverse phase HPLC, to obtain a profile (purity 27.3%) as shown in FIG. 2.

### EXAMPLE 2

### Solid Phase Synthesis of Salmon Calcitonin

a) Peptide Condensation

250 mg of 4-(2,4-dimethoxyphenyl-Nsc-aminomethyl)phenoxy resin (1.0 meq, NH₂/g) was washed 3 times with 5 ml DMF, added with 33% piperidine/DMF and reacted for 20 minutes, followed by washing 3 times with 5 ml of DMF and then 3 times with 5 ml of DCM, to synthesize the peptide according to the following protocol:

1. prewashing: 33% piperidine/DMF, 4 ml:0.5 minutes

2. deprotection: 33% piperidine/DMF, 4 ml:15 minutes

3. washing: DMF, 3×(4 ml: 1 minute); DCM, 3×(4 ml: 1 minute)

4. acylation: N_{α}-Nsc-amino acid as described below, 0.5 mmol; benzotriazolyl-1-oxo-(dimethylamino)phosphonium hexafluorophosphate, 0.5 mmol; 1-hydroxybenzotriazol, 0.5 mmol; diisopropylimide, 1 mmol; DCM, 3 mmol: 10 minutes (40° C.)

5. washing: DCM, 5×(4 ml: 1 minute)

N_{α}-Nsc-amino acids were used for synthesis, according to the following sequence:

Nsc-Pro-OH, Nsc-Thr(tBu)-OH, Nsc-Gly-OH, Nsc-Ser(tBu)-OH, Nsc-Gly-OH, Nsc-Thr(tB u)-OH, Nsc-Asn(trt)-OH, Nsc-Thr(tBu)-OH, Nsc-Arg(pbf)-OH, Nsc-Pro-OH, Nsc-Tyr(tBu)-OH, Nsc-Thr(tBu)-OH, Nsc-Gln(trt)-OH, Nsc-Leu-OH, Nsc-Lys(Boc)-OH, Nsc-His(trt)-OH, Nsc-Leu-OH, Nsc-Glu(OtBu)-OH, Nsc-Gln(trt)-OH, Nsc-Ser(tBu)-OH, Nsc-Leu-OH, Nsc-Lys(Boc)-OH, Nsc-Gly-OH, Nsc-Leu-OH, Nsc-Val-OH, Nsc-Cys(acm)-OH, Nsc-Thr(tBu)-OH, Nsc-Ser(tBu)-OH, Nsc-Leu-OH, Nsc-Asn(trt)-OH, Nsc-Ser(tBu)-OH, Nsc-Cys(acm)-OH.

The assembly of peptidyl-resin polymer having the target amino acid sequence was prepared and dried under vacuum in the presence of phosphorous pentoxide for 24 hours.

b) Cysteine Partial Deprotection and Cysteine Bridge Formation

The dried peptidyl-resin polymer was swelled in 10 ml of DMF for 30 minutes, added with 2.5 mmol iodine and stirred at room temperature for 2 hours. Thereafter, 3 g of ascorbic acid was added thereto and stirred for 1 hour, followed by stopping the reaction. Then, the reaction was filtered, washed 3 times with DMF, to yield the bridge-formed Nsc-sacatonin-polymer, which was then treated with 33% piperidine/DMF (4 ml) for 20 minutes, and washed with DMF and DCM, ethanol, ether, and hexane.

c) Deprotection and Peptide Detachment

The sacatonin polymer was added to 5 ml of a mixture comprising 95% trifluoroacetic acid, 2.5% water and 2.5% triisopropylsilane and shaken at room temperature for 60 minutes. Such reaction was filtered, washed with 2 ml of trifluoroacetic acid, and the filtrate combined with washings was diluted with 100 ml of cold absolute ether. The precipitate was filtered, washed with ether and dried in vacuo, giving 810 mg of crude sacatonin (yield: 92%).

d) Analysis

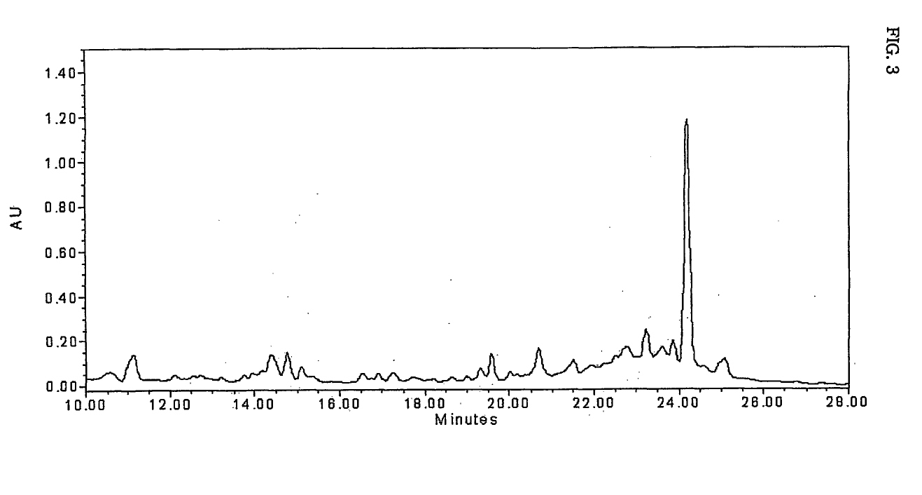

The crude sacatonin fraction was dissolved in 1 ml of 1% acetic acid/purified water and analyzed by reverse phase HPLC using DISOGEL RP-C18 (Disogel, Japan 4.5×250 ml), to obtain a profile (purity 47%) as shown in FIG. 3. As such, pure peptide containing portion was collected and analyzed using a mass analyzer, to obtain the desired M+H 3432.

### COMPARATIVE EXAMPLE 2

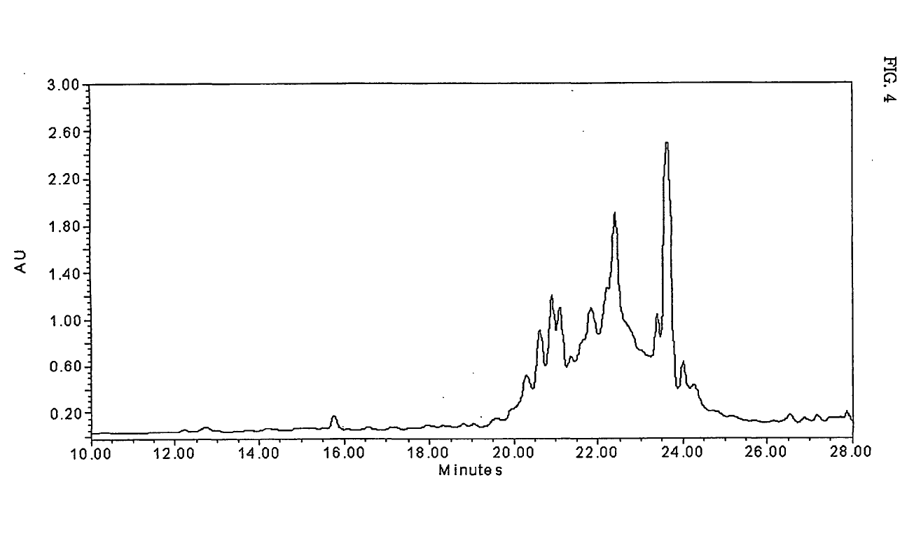

According to the solid phase peptide synthesis using Fmoc amino acids, except that acylation reaction was performed using the following Fmoc-amino acids as the amino acid and DMF as the acylation solvent at room temperature for 1 hour, 755 mg of crude sacatonin (yield:85.8%) was produced, which was then analyzed by reverse phase HPLC, to obtain a profile (purity 14.5%) as shown in FIG. 4.

Fmoc-Pro-OH, Fmoc-Thr(tBu)-OH, Fmoc-Gly-OH, Fmoc-Ser(tBu)-OH, Fmoc-Gly-OH, Fmoc-Thr(tBu)-OH, Fmoc-Asn(trt)-OH, Fmoc-Thr(tBu)-OH, Fmoc-Arg(pbf)-OH, Fmoc-Pro-OH, Fmoc-Tyr(tBu)-OH, Fmoc-Thr(tBu)-OH, Fmoc-Gln(trt)-OH, Fmoc-Leu-OH, Fmoc-Lys(Boc)-OH, Fmoc-His(trt)-OH, Fmoc-Leu-OH, Fmoc-Glu(OtBu)-OH, Fmoc-Gln(trt)-OH, Fmoc-Ser(tBu)-OH, Fmoc-Leu-OH, Fmoc-Lys(Boc)-OH, Fmoc-Gly-OH, Fmoc-Leu-OH, Fmoc-Val-OH, Fmoc-Cys(acm)-OH, Fmoc-Thr(tBu)-OH, Fmoc-Ser(tBu)-OH, Fmoc-Leu-OH, Fmoc-Asn(trt)-OH, Fmoc-Ser(tBu)-OH, Fmoc-Cys(acm)-OH.

### INDUSTRIAL APPLICABILITY

As described above, the inventive peptide synthesis method using Nsc-amino acids is advantageous in light of preparation of peptides having high yield and high purity within a short time period by performing the acylation reaction at a high temperature with the use of dichloromethane as the acylation solvent. Further, Nsc-amino acids can be stored in a solution state dissolved in dichloromethane, whereby Nsc-amino acids need not be prepared for each synthesis of peptide and can be stored at room temperature without freezing.

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

1. A method of preparing a peptide, comprising the following steps of:
forming a protected aminoacyl-polymer onto an insoluble carrier through a free carboxyl group of a protected amino acid;
deprotecting the protected aminoacyl-polymer;
acylating a protected amino acid monomer to a free amino group of the deprotected polymer;
repeating the deprotection and the acylation steps until a target amino acid sequence-containing peptide is synthesized; and
detaching the synthesized peptide from the insoluble carrier after deprotection of a terminal protective group in the synthesized peptide,
wherein, as the protected amino acid, 2-(4-nitrophenylsulfonyl)ethoxycarbonyl-amino acids (Nsc-amino acids) are used, and the acylating step is performed using a dichloromethane solvent in the temperature range of from 20 to 50° C.

2. The method as defined in claim 1, wherein the acylating step is conducted for from 5 minutes to 1 hour.

3. A method of storing Nsc-amino acids comprising the step of storing 2-(4-nitrophenylsulfonyl)ethoxycarbonyl-amino acids (Nsc-amino acids) in a solution state dissolved in dichloromethane.
